(19) United States
(12) Patent Application Publication
(10) Pub. No.: US 2025/0367019 A1
(43) Pub. Date: Dec. 4, 2025

(54) NONINVASIVE UNISEX EXTERNAL CATHETER

(71) Applicant: DrySense a California corporation, Los Angeles, CA (US)

(72) Inventors: Victor Musoke, Van Nuys, CA (US); Ricardo Borja, Trois-Rivières (CA)

(21) Appl. No.: 19/287,690

(22) Filed: Jul. 31, 2025

Related U.S. Application Data
(63) Continuation-in-part of application No. 18/117,389, filed on Mar. 3, 2023.

(51) Int. Cl. A61F 5/44; A61F 5/441; A61F 5/443

(52) U.S. Cl. CPC A61F 5/4405; A61F 5/441; A61F 5/443

(57) ABSTRACT

A non-invasive unisex external catheter is provided. The device features a dressing element configured for attachment to a user and a pliable, concave body that rests upon it. A flange secures the perimeter of the body to the dressing element, creating a sealed enclosure over the user's genital area. Inside the body, a suction element, which may be a tube with at least one drainage hole, collects and guides urine away for disposal. The dressing element and flange may utilize peelable adhesive layers to ensure a secure, leak-proof attachment. The catheter's pliable construction allows it to conform to diverse body types, enhancing user comfort and offering a hygienic alternative to invasive methods.

### CROSS-REFERENCE TO RELATED APPLICATIONS

This Continuation in Part CIP) patent application claims the benefit of U.S. application Ser. No. 18/117,389 filed on Mar. 3, 2023, and from PCT No. PCT/US24/18276 filed on Mar. 3, 2024, all of which are incorporated by reference herein in their entirety.

### BACKGROUND

Current measures for bladder control are invasive, leave users vulnerable to urinary tract and skin infections, require frequent change management, and are prone to leakages.

### FIELD OF INVENTION

Embodiments of the invention are related to noninvasive urinary evacuation and more particularly concerns an external catheter for both men and women.

### SUMMARY OF THE INVENTION

A noninvasive, unisex external catheter will now be described in accordance with one or more embodiments of the invention. The following exemplary description provides numerous specific details to provide a more thorough understanding of embodiments of the invention. Furthermore, although steps or processes are set forth in an exemplary order to provide an understanding of one or more systems and methods, the exemplary order is not meant to be limiting. One of ordinary skill in the art will recognize that in some instances it is possible to perform the steps or processes in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the various embodiments of the invention described herein, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render effective, but these are intended to cover the application or implementation.

The term “first”, “second” and the like, herein do not denote any order, quantity or importance, but rather are used to distinguish one element from another, and the terms “a” and “an” herein do not denote a limitation of quantity, but rather denote the presence of at least one of the referenced items.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration. Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Disclosed in this application is an external catheter comprising a) a body, wherein the body is a hollow concave shape, b) a lip, wherein the lip is attached around a perimeter of the body, c) a silicone layer, wherein the silicone layer is a single layer of the body, d) a drain element, wherein the drain element is configured for urine drainage, e) an outlet, wherein the outlet covers the drain element, f) a fiber element, wherein the fiber element is attached to the body, wherein the fiber element abuts to the drain element, g) a collection region, wherein the collection region abuts to the fiber element, h) a vent, wherein the vent abuts to the body, and i) a valve, wherein the valve is attached to the vent. In addition, the external catheter is unisex. The silicone layer c) enables the body to be elastic. This elasticity enables the body to contour to a plurality of human bodies. The collection region g) wherein the fiber element guides a stream of urine to the drain element, wherein the stream of urine is collected for disposal. In addition, the valve i) is configured to allow air inside the body, wherein the valve does not allow liquid outside the body.

One or more embodiments of the invention is an external catheter comprising a) a body, wherein the body is a hollow concave shape, b) a lip, wherein the lip is attached around perimeter of the body, c) an adhesive element, wherein the adhesive element is attached to the lip, wherein the lip and the adhesive element form a border such that the border enables a sealed attachment to a human body, d) a silicone layer, wherein the silicone layer is a single layer of the body, e) a drain element, wherein the drain element is configured for urine drainage f) an outlet, wherein the outlet covers the drain element, g) a fiber element, wherein the fiber element is attached to the body, wherein the fiber element abuts to drain element, h) a collection region, wherein the collection region abuts to the fiber element, i) a vent, wherein the vent abuts to the body, and j) a valve, wherein the valve is attached to the vent. In addition, the external catheter is unisex. The silicone layer d) wherein the silicone layer enables the body to be elastic, wherein the elasticity enables the body to contour to a plurality of human bodies. The collection region h) wherein the fiber element guides a stream of urine to the drain element, wherein the stream of urine is collected for disposal. In addition, the adhesive element c) wherein the adhesive element prevents urine from leaking outside the body during urination. The valve j) wherein the valve is configured to allow air inside the body, wherein the valve does not allow liquid outside the body.

One or more embodiments of the invention is an external catheter comprising a) a body, wherein the body is a hollow concave shape, b) a lip, wherein the lip is attached around a perimeter of the body, c) a silicone layer, wherein the silicone layer is a single layer of the body, d) a drain tube, wherein the drain tube is configured for urine drainage, e) an outlet, wherein the outlet covers the drain tube, f) a cotton material, wherein the cotton material is attached to the body, wherein the cotton material abuts to drain tube, g) a collection region, wherein the collection region abuts to the cotton material, h) a vent, wherein the vent abuts to the body, i) a valve, wherein the valve is attached to the vent and j) an attachment element, wherein the attachment element is configured to hold the body, wherein the attachment element is adjustable. In addition, the silicone layer c) wherein the silicone layer enables the body to be elastic, wherein the elasticity enables the body to contour to a plurality of human bodies. The collection region g), wherein the cotton material guides a stream of urine to the drain tube, wherein the stream of urine is collected for disposal. The cotton material f), wherein the cotton material is placed inside the outlet. The valve i), wherein the valve is configured to allow air inside the body, wherein the valve does not allow liquid outside the body.

One or more embodiments of the invention is an external catheter comprising a) a body, wherein the body is a hollow concave shape, b) a lip, wherein the lip is attached around a perimeter of the body, c) a silicone layer, wherein the silicone layer is a single layer of the body, d) a drain tube, wherein the drain tube is configured for urine drainage, e) an outlet, wherein the outlet covers the drain tube, f) a cotton material, wherein the cotton material is attached to the body, wherein the cotton material abuts to drain tube, g) a collection region, wherein the collection region abuts to the cotton material, h) a vent, wherein the vent abuts to the collection region, i) a valve, wherein the valve is attached to the drain element, j) an attachment element, wherein the attachment element is configured to hold the body, wherein the attachment element is adjustable, and k) an underwear, wherein the underwear is configured with the attachment element to hold the body, wherein the underwear is unisex. In addition, the silicone layer c), wherein the silicone layer enables the body to be elastic, wherein the elasticity enables the body to contour to a plurality of human bodies. The collection region g), wherein the cotton material guides a stream of urine to the drain tube, wherein the stream of urine is collected for disposal. The cotton material f), wherein the cotton material is placed inside the outlet. The valve i), wherein the valve is configured to allow air inside the body, wherein the valve does not allow liquid outside the body.

One or more embodiments of the invention is a non-invasive unisex external catheter comprising, a) a dressing element configured to attach to a user, b) a body which is attached to the dressing element, c) a flange attached around a perimeter of the body, where the flange is configured to attach to the dressing element, and d) a suction element, where the suction element is inserted inside the body, the suction element having at least one hole for urine drainage. It should be noted that the suction element d), is configured to guide a stream of urine outside the body b), and the stream of urine is collected for disposal. The flange c) has a peelable layer, wherein an adhesive layer remains once the peelable layer is removed. The dressing element a), can also have a peelable layer, where the adhesive layer remains once the peelable layer is removed. Also, the flange has a slit configured to allow the suction element to be inserted inside the body. It should be noted that the body b) has a concave shape. Also, the dressing element a), is configured to attach to the user via an adhesive. The dressing element and or flange, can be a breathable material. Also, the body can be elastic or pliability and enables the body to contour to a plurality of the users.

One or more embodiments of the invention is a non-invasive unisex external catheter comprising, a) a dressing pad, configured to attach to a user via an adhesive, b) a body having a hollow concave shape, configured to sit on the dressing pad, c) a flange, attached around a perimeter of the body, where the flange is attached to the dressing pad, d) a slit, located on the flange, and e) a tube, inserted inside the body via the slit, and the tube having at least one hole for urine drainage. Also, the urine is drained through at least one hole for disposal outside the body. It should be noted that the flange c) has a peelable layer, where an adhesive remains once the peelable layer is removed. The dressing pad a), also can have a peelable layer, where the adhesive remains once the peelable layer is removed. The flange c) has a slit configured to allow the tube to be inserted inside the body. It should be noted that the dressing pad and the flange can be breathable material. In addition, the body b), can be pliable or elastic, where the pliability enables the body to contour to a plurality of users.

One or more embodiments of the invention is a method of attaching a non-invasive unisex external catheter on a user comprising a) peeling off a peelable layer of a dressing element, b) attaching the dressing element on a user by positioning the dressing element around a genital area of the user, c) pressing the dressing element against the user to establish a seal around the genital area of the user, d) peeling off a peelable layer of a flange, e) attaching bottom end of the flange to the dressing element, f) placing body over the genital area of the user, g) pressing on bottom end of the flange, where the pressing the bottom end of flange enables a seal between the dressing element and the bottom end of flange, g) wrapping suction element around the user's genitals, where the suction element forms a loop around the user's genitals, h) inserting a suction element through a slit located on the flange thereby placing the suction element inside the body, i) attaching top end of the flange to the dressing element, and j) pressing on top end of the flange, where the pressing the top end of flange enables a seal between the dressing element and the top end of flange. It should be noted that the flange has a peelable layer wherein an adhesive layer remains once the peelable layer is removed. In addition, the dressing element has a peelable layer, where the adhesive layer remains once the peelable layer is removed.

References. The description of the figures provided herein contain references to each depicted component. A list of these components described in the context of each figure is provided below for easy reference.

| | |
|---|---|
| 100 | Body |
| 102 | Lip |
| 104 | Adhesive Element |
| 106 | Drain Element |
| 108 | Fiber Element |
| 110 | Outlet |
| 111 | Vent |
| 112 | Collection Region |
| 113 | Valve |
| 114 | Fiber Element Support |
| 200 | Silicone Layer |
| 500 | Attachment Element |
| 600 | Underwear |
| 702 | Suction Element |
| 703a | Top End of Suction Element |
| 703b | Bottom End of Suction Element |
| 704 | Slit |
| 706 | Flange |
| 708 | Flange Peelable Layer |
| 709 | Adhesive Layer |
| 710 | Dressing Element |
| 712 | Dressing Element Peelable Layer |
| 714 | Connector Element |
| 802 | Perforated Holes |
| 1002 | Peeling Off the Peelable Layer of the Dressing Element |
| 1004 | Attaching the Dressing Element To Human Skin |
| 1006 | Pressing Around the Dressing Element |
| 1008 | Peeling Off the Peelable Layer of the Flange |
| 1010 | Attaching the Bottom End of the Flange to the Dressing Element |
| 1012 | Placing the Body over the Human Genital |
| 1014 | Pressing Down on the Bottom End of the Flange |
| 1016 | Placing the Suction Element Around the User's Genitals Inside |
| | the Body |
| 1017 | Inserting the Suction Element Through the Slit on the Flange |
| 1018 | Attaching the Top End of the Flange to the Dressing Element |
| 1020 | Pressing Down on the Top portion of the Flange |

### BRIEF DESCRIPTION OF THE DRAWINGS

FIG. 1 illustrates the front and back view of the external catheter.

FIG. 2 illustrates the side view of the external catheter.

FIG. 3 illustrates an exploded side view of the external catheter.

FIG. 4 illustrates a side cross sectional view of the external catheter.

FIG. 5 illustrates the external catheter attached to an attachment element.

FIG. 6 illustrates the catheter attached to an underwear.

FIG. 7 illustrates an Exploded view of the external, non-invasive, unisex catheter.

FIG. 8 illustrates the close-up view of the perforated suction element.

FIG. 9 illustrates a front view of the external, non-invasive, unisex catheter.

FIG. 10 illustrates a method of attaching the external, non-invasive, unisex catheter to a user.

### DETAILED DESCRIPTION

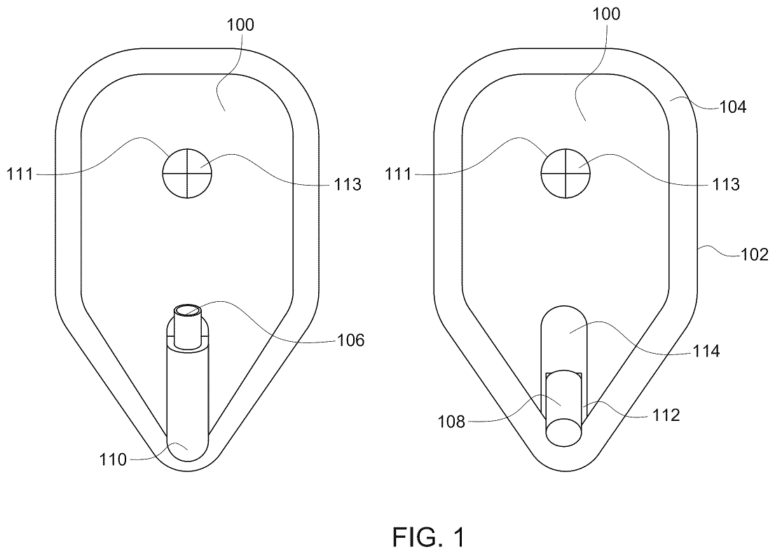

FIG. 1 illustrates an external catheter which can contour to many different types of human bodies or users in a noninvasive way as it is configured in accordance with one or more embodiments of the invention. In this illustrated example, the external catheter comprises a body (100), a lip (102), an adhesive element (104), a drain element (106), a fiber element (108), an outlet (110), a vent (111) a collection region (112), a valve (113) and a fiber element support (114).

The body (100) can be, but is not limited to, a flexible hollow concave non condom structure such that is flexible in nature allowing the body (100) to contour or mold to a plurality of human body types. Utilizing a flexible structure allows the external catheter to accommodate men and women of different sizes and shapes. The body (100) of the external catheter covers or encloses a human genital area by covering a human penis or a human vagina. One end of the external catheter is either left open-ended, to allow drainage into a toilet, or it is attached to a bag to collect urine. In this instance the drain element (106) is attached to the body (100). The drain element can be but is not limited to a drain pipe or a drain tube, or any equivalent structure that guides the urine to move into an external urine collection bag or receptacle. The drain element (106) is placed inside a harder structure called the outlet (110). The drain element (106) is configured for either supine or standing urine drainage. The outlet (110) holds the drain element (106) in place as well as the fiber element (108) which can be made of cotton, cotton material, or any type of fabric material or any other material able to perform the intended purpose. The vent (111) and the valve (113) work in conjunction to allow air from outside the body (100) to come into the body (100), but the valve (113) prevents liquid from exiting the body (100). The valve (113) can be made by a check valve or an extra membrane layer or silicone or can be mechanical in structure. The vent (111) is configured to decrease vacuum pressure in the body (100) so the contents of the body (100) can be voided out with more ease. The function of the fiber element (108) is to guide a stream of urine to the outlet (110). The fiber element (108), outlet and drain element (106) or an equivalent structure are configured to keep urine away from the body (100). In one or more embodiments of the invention, the fiber element (108), outlet (110) and drain element (106) or an equivalent structure are each curved and attached to the hallow concave shape of the body (100) of the external catheter. However, before urine from the user enters the fiber element (108), outlet (110) and drain element (106), the urine is first collected in collection region (112) at the bottom section of the body (100). The concave shape of the body (100) enables urine to flow downward towards the collection region (112) where urine flows into the fiber element (108), outlet (110) and drain element (106). The fiber element is supported by the fiber support element (114), which allows the fiber element (108) to stay in place and not move around during urination, which could cause urine leakage outside the body (100). In addition, the fiber support element (114) covers the interior of the body (100) so in case a human is in a lateral position or any other position, the body (100) will collect liquid and will not allow for leakage of the liquid.

In one exemplary implementation the body (100) contains a lip (102) which forms the border of the body (100). Attached to the lip (102) is an optional layer referred to as an adhesive element (104). The adhesive element (104) could be but is not limited to an adhesive border or an adhesive strip or an adhesive perimeter or any equivalent structure that performs the intended purpose of affixing the body to the user. The adhesive element (104) can be made from but is not limited to a Mepilex brand or an equivalent material that performs the intended purpose of adhering the body to the user. The adhesive element (104) is designed to form a flat, sealed surface around the lip (102) of the body (100), enabling the body (100) to securely attach to a surface such as human skin, while effectively preventing urine leakage during urination.

Traditional catheters are inserted into a penis or a vagina, to drain, of fluids such as urine and therefore can be classified as an invasive since invasive procedures are where deliberate access to the body is gained via an incision, percutaneous puncture, or where medical instruments are used in addition to the puncture needle, or instrumentation via a natural orifice. The external catheter disclosed here does not require any incision, puncture, or any type of invasive methods. Instead, the human penis or vagina sits inside the concave body (100) of the external catheter. When there is urination, the urine will flow downward towards the collection region (112) where urine is collected and subsequently sent down via gravity into the fiber element (108), outlet (110) and drain element (106). The drain element (106) can be connected to a bag or an equivalent structure where urine can be collected.

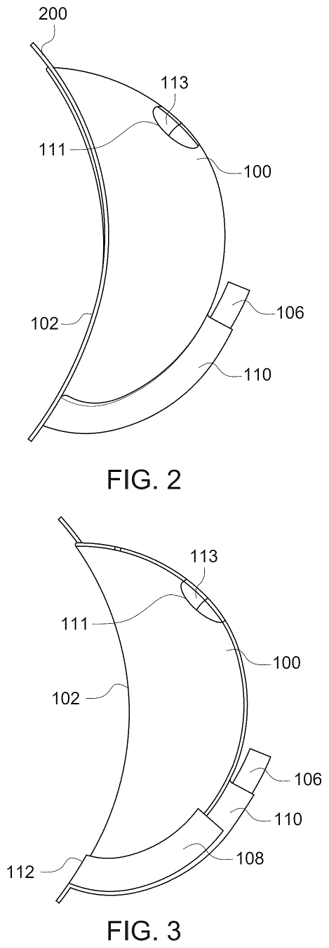

FIG. 2 illustrates the side view of the external catheter in accordance with one or more embodiments of the invention. In this illustrated example, the external catheter comprises a body (100), a lip (102), a drain element (106), an outlet (110), a vent (111), a valve (113), and a silicone layer (200).

The side view of the body of the external catheter illustrates the concave shape of the body (100) as well as the thickness of the lip (102) which is around the body (100). Also shown in the figure is the outlet (110) which encapsulates the drain element (106). The body (100) of the external catheter is elastic such that it can contour to different humans with different shapes and size genitals. Also shown in FIG. 2 is the silicone layer (200) of the body (100). The body (100) can be made from one or more silicone layers because the optional additional silicone layers provide an additional center thickness of the body (100), where the extra center thickness keeps urine away from the body (100). In addition, the additional thickness at the center of body (100) keeps the fiber element (108) and urine elevated away from the human user. Silicone is a polymer made up of siloxane and can vary in consistency from liquid to gel to rubber to hard plastic depending on how much layering is present. The vent (111) and the valve (113) work in conjunction to allow air to come into the body (100) but prevent liquid from exiting the body (100). The vent (111) is located on the exterior of the body (100) and the valve (113) is attached to the vent (111). The single silicone layer (200) enables the body (100) to be flexible due to the elastic characteristics of silicone and allows the body (100) to contour to different body types.

FIG. 3 illustrates a transparent side view of the external catheter in accordance with one or more embodiments of the invention. In this illustrated example, the external catheter comprises a body (100), a lip (102), a drain element (106), a fiber element (108), an outlet (110), a vent (111), a collection region (112) and a valve (113).

In this side view of the body (100) of the external catheter, the internal elements of the external catheter can be seen. For example, the fiber element (108) is located inside the outlet (110) abutting the collection region (112) on one end to the drain element (106) on the other end. In addition, the fiber element (108) is typically cotton, a section of a fabric material or any equivalent material that serves the intended purpose of guiding a stream of urine to the drain element (106), is connected to the drain element (106). The outlet (110), fiber element (108) and the drain element (106) each form a curved structure that sits on the surface of the concave body (100) of the external catheter. Also shown in FIG. 3 is the lip (102) of the body (100). The lip (102) attaches to an optional adhesive element (104). The lip (102) and the adhesive element (104) enable a more secure contact between the body (100) of the external catheter and the human user.

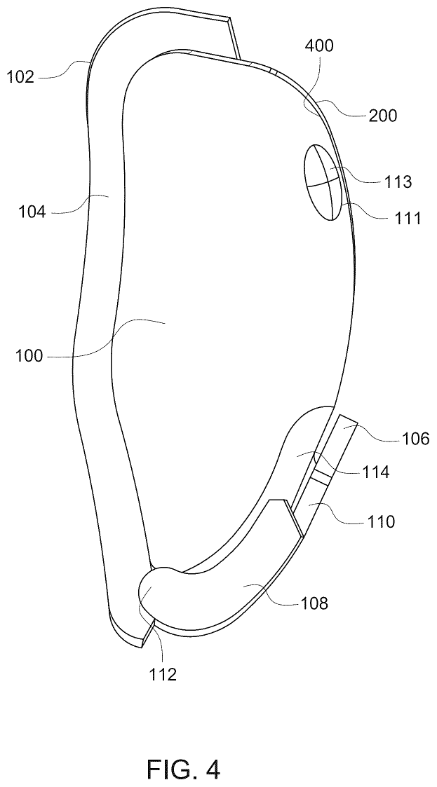

FIG. 4 illustrates a cross-section side view of the external catheter in accordance with one or more embodiments of the invention. In this illustrated example, the external catheter comprises a body (100), a lip (102), a drain element (106), a fiber element (108), an outlet (110), a vent (111), a collection region (112), a valve (113), a fiber support element (114), and a silicone layer (200),

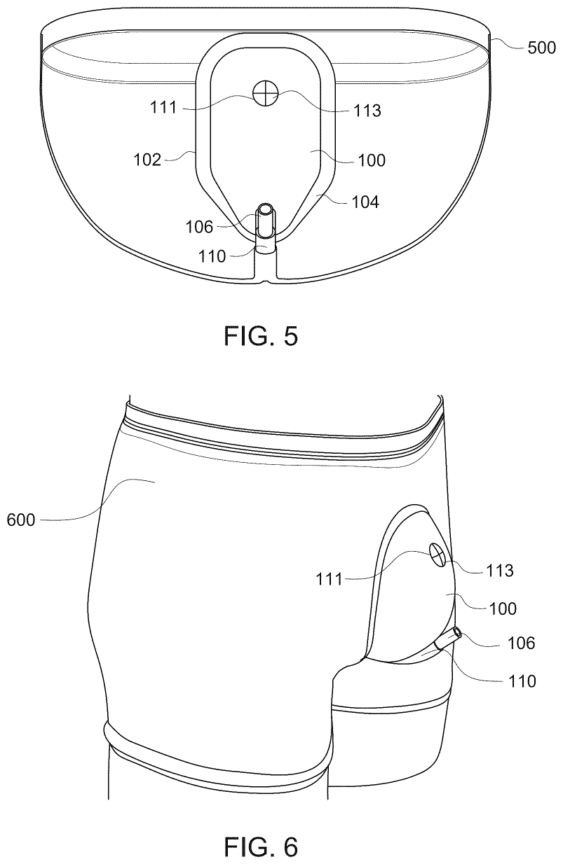

FIG. 5 illustrates an external catheter inside an attachment element (500) in accordance with one or more embodiments of the invention. In this illustrated example, the external catheter and an attachment element comprises a body (100), a lip (102), a drain element (106), an outlet (110), a vent (111), a valve (113), and an attachment element (500).

In this embodiment of the invention, the body (100) of the external catheter is inside an attachment element (500) such that the attachment element (500) holds the body (100) in place and also allows the user to wear the attachment element (500). The attachment element (500) ties around the user's waist and enables the user to put their legs through the bottom section of the attachment element (500), where the attachment element securely fits around the user's waist and legs. The body (100) is positioned in the attachment element (500) such that the concave body (100) of the external catheter fits over the user's penis or vagina. The attachment element (500) is configured for either supine or standing urine drainage. The attachment element (500) allows for the external catheter to stay in place while the user is walking, sitting, or going up stairs or any other equivalent action requiring movement. The attachment element (500) can be adjustable such that it can be configured to fit around different people of varying shapes and sizes. The adjustment can be accomplished by velcro, or elastic snappable velcro with different notches for different waist sizes or a belt-like material with adjustable notches or any equivalent structure that performs the adjustability function.

FIG. 6 illustrates an external catheter attached to an underwear (600) in accordance with one or more embodiments of the invention. In this illustrated example, the external catheter and an underwear (600) comprises a body (100), a drain element (106), an outlet (110), a vent (111), a valve (113), and the attachment element (500).

In this embodiment of the invention the body (100) of the external catheter is attached to an underwear (600) such that the underwear (600) and the attachment element (500) work in conjunction to hold the body (100) of the catheter in place and also allows the user to wear the underwear (600). The underwear (600) could be made for either for men or women. Underwear (600) could also be but not limited to shorts, men undergarment, pantyhose, women's undergarments, or any equivalent clothing that performs the intended purpose of an underwear (600). The body (100) is positioned in the underwear (600) such that the concave body (100) of the external catheter fits over the user's penis or vagina. The underwear (600) and the external catheter in this embodiment is configured for either supine or standing urine drainage. The underwear (600) allows for the external catheter to stay in place while the user is walking, sitting, or going up stairs.

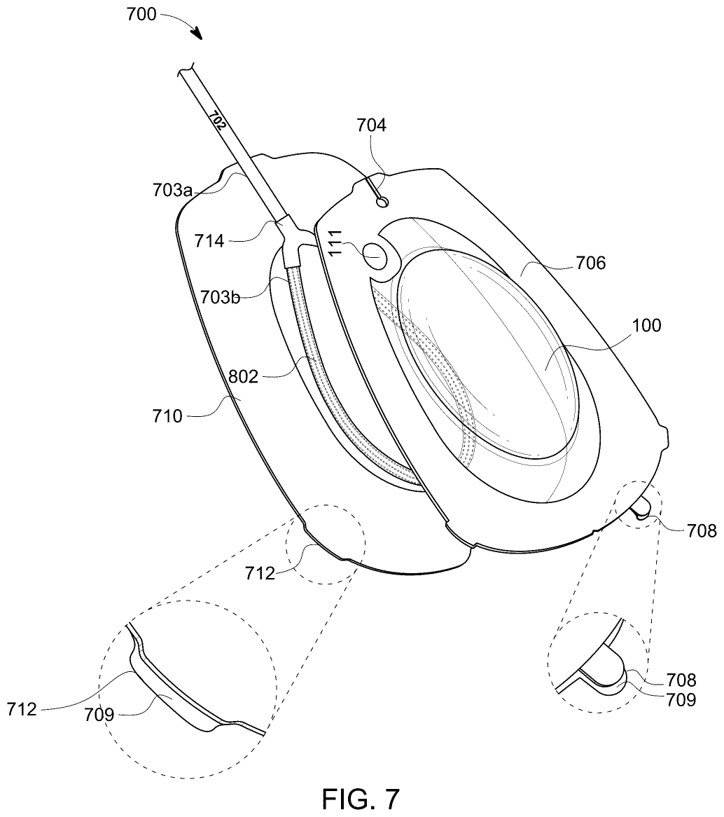

FIG. 7 illustrates an exploded view of the external, non-invasive, unisex catheter in accordance with one or more embodiments of the invention.

The external catheter in FIG. 7 can contour to many different types of human genitals having noninvasive capabilities as configured in accordance with one or more embodiments of the invention. In this illustrated example, the external catheter comprises a body (100), a suction element (702), a slit (704), a flange (706), a flange peelable layer (708), a dressing element (710), a vent (111), a dressing element peelable layer (712), a connector element (714), which connects the top end (703a) and the bottom end (703b) of the suction element (702) and the perforated holes (802) which are located throughout the suction element (702).

The body (100) can be but is not limited to a flexible or pliable hollow concave non condom structure that is able to contour or mold to a plurality of human bodies or users. The body (100) comfortably captures fluids without causing discomfort to the user. The device is functional on various types of human bodies and usable by women and men of different sizes and shapes. The body (100) of the external catheter covers or encloses a human penis or vagina.

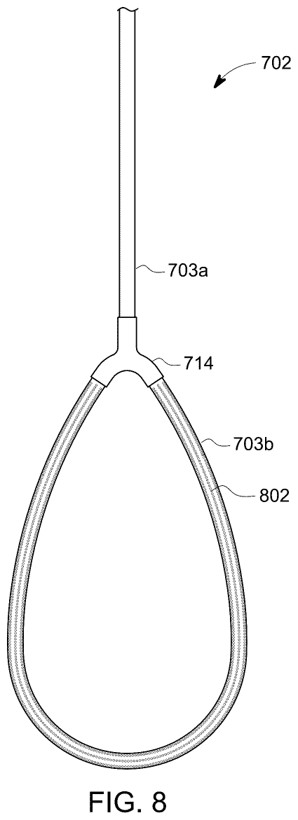

The top end (703a) of the suction element (702) is either left open-ended, to allow drainage into a toilet, or the suction tube is attached to a bag or suction device to collect urine. It should be noted that the suction element (702), which can be a tube, or a hollow structure, or can be a continuous structure without a connector element (714). The suction element (702) can be made by the connection of a top end (703a) and a bottom end (703b) via a connector element (714) as shown in FIGS. 7 and 8.

In this embodiment of the invention, the flange (706) is attached around the perimeter of the body (100). It should be noted that the flange (706) has a peelable layer (708) located on the bottom surface of the flange (706) which can be peeled off allowing the flange (706) and body (100) to be attached to the dressing element (710) via an adhesive. Once the peelable layer of the flange (708), is peeled off, what remains in the place of the peelable layer is an adhesive layer (709). The dressing element (710) can be but is not limited to a dressing pad, skin shield or a donut shaped structure, which can adhere to skin despite the presence of hair and withstanding the accumulation of fluid without significant leakage. The dressing element (710) and the flange (706) are both made from breathable materials, which means the dressing element (710) and flange (706) allow air and moisture to pass through, facilitating evaporation and helping to regulate body temperature and prevent overheating and discomfort by allowing sweat on the skin to evaporate. The dressing element (710) has a peelable layer (712) located on the bottom side, which allows the dressing element (710) to be attached to a human user via an adhesive. Once the peelable layer of the dressing element (710) is peeled off, what remains in the place of the peelable layer is an adhesive layer (709). Readers should note that any type of adhesive suitable for contact with human skin can be used on the adhesive layer (709). The vent (111), which can be left open or closed, allows air from outside the body (100) to come into the body (100). The vent (111) is configured to decrease vacuum pressure in the body (100) so the contents of the body (100) can be evacuated with more ease. The flange (706) has a slit (704) which allows the suction element (702) to be inserted inside the body (100). The suction element (702), which can be but is not limited to a tube, pipe or an equivalent hollow structure, is perforated with holes throughout the suction element (702) in one exemplary configuration. The concave and hollow shape of the body (100) enables liquid to flow downward towards the bottom portion of the suction element (702) where urine flows inside the suction element (702) via the perforated holes (802). The suction element (702) can be attached to a suction device that can pull the urine through the suction element (702) via the perforated holes (802) and collect urine outside the body (100) where it can be collected or discarded.

In case a human is excreting urine in a lateral or any other position, the body (100) in conjunction with the flange (706), the suction element (702) and the dressing element (710) will collect liquid and prevent substantial leakage of urine.

FIG. 8 illustrates the suction element in accordance with one or more embodiments of the invention.

The suction element (702) FIG. 8 comprises perforated holes (802) which can be located throughout the suction element (702). It should be noted that in FIG. 8, the suction element (702), which can be a tube or equivalent structure, can be but is not limited to a loop which evacuates fluids. The suction element (702) which lies inside the body (100) can be alternatively shaped as an oval, ellipse, loop, spheroid, or rectangle or just a straight line. In addition, the body (100) can be elastic or pliable thereby allowing it to contour or mold to different types of human shapes and sizes without tearing or breaking. Also shown is the top end (703a) and the bottom end (703b) of the suction element (702). It should be noted that the suction element (702), can be either a continuous structure where the suction element (702) is one continuous form without a connecting element (714) or it can be made by two or more pieces wherein a top end (703a) and a bottom end (703b) are connected via a connector element (714) forming the suction element (702) as shown in FIGS. 7 and 8. In addition, a wick sleeve or just a sleeve could be added around the suction element (702) holding the suction element (702) in place and restricting the suction element (702) from excessive movement when the user is moving.

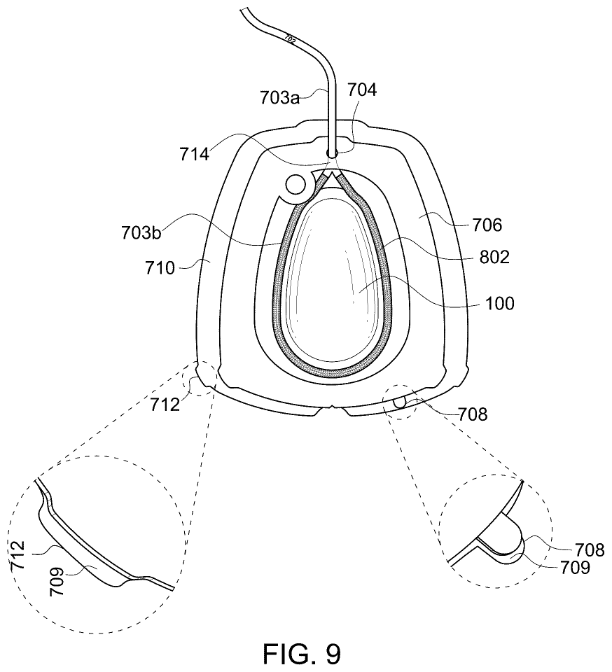

FIG. 9 illustrates the front view of external, non-invasive, unisex catheter in accordance with one or more embodiments of the invention.

The external catheter in FIG. 9 can contour to many different types of human bodies or users in a noninvasive manner as the external catheter is configured in accordance with one or more embodiments of the invention. In this illustrated example, the external catheter comprises a body (100), a suction element (702), a slit (704), a flange (706), a flange peelable layer (708), a dressing element (710), a dressing element peelable layer (712) a vent (111) and the perforated holes (802) located throughout the suction element (702).

The body (100) can be made from one or more polymer layers because the optional additional polymer layers provide an additional center thickness of the body (100), where the extra center thickness can improve keeping urine away from the body (100). The polymer layers described above can be either elastic or pliable in texture depending on how much layering is present. The vent (111) allows air to come into the body (100) but prevents liquid from exiting the body (100). The vent (111) is located on the exterior of the body (100).

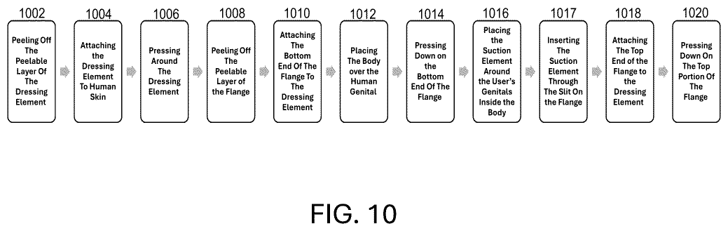

FIG. 10 illustrates the method of placing the external, non-invasive, unisex catheter on a human user in accordance with one or more embodiments of the invention.

The first step (1002) includes peeling the peelable layer (712) of the dressing element (710), which is located on the bottom of the dressing layer and attaching (1004) the dressing element (710) on a human user such that the dressing element (710) is positioned around the user's genital. The attachment occurs via an adhesive, which is located on the bottom surface of the dressing element (710). Step (1006) requires pressing around the dressing element (710) to seal the dressing element (710) all around the user's genital area ensuring a tight or firm seal formed between the dressing element (710) and the skin around the user's genitals. Step (1008) requires peeling of the peelable layer (708), which is located on the bottom surface of the flange (706). As previously discussed, the flange (706) is attached to the body (100). The next step requires attaching (1010) the flange (706) to the dressing element (710). The attachment is achieved via an adhesive. The next step requires placing (1012) the body (100), over the user's genitals such that the genitals are inside the hollow, concave shape of the body (100). Step (1014) requires pressing down on the bottom portion of the flange (706) to make sure that there is a firm or tight seal between the bottom portion of the flange (706) and the dressing element (710). Step (1016) requires placing the suction element (702) around the user's genitals inside the body (100) and then inserting (1017) the suction element (702) through the slit (704) such the top end (703a) of the suction element (702) can be attached to an external suction device where the urine is collected and discarded. The bottom end (703b) of the suction element (702) must be positioned inside the body (100) such that the perforated holes (802) of the suction element (702) can collect urine inside the body (100). The next step (1018) requires the top of the flange (706) to be attached to the dressing element (710). Attachment occurs via an adhesive which is located on the bottom surface of the flange (706). Finally, the next step (1020) requires pressing down on the top end of the flange (706) in order to seal the flange (706) to the dressing element (710). To achieve a tight or firm seal between the flange (706) and the dressing element (710), the user should seal from the top of the flange (706) and move down the flange (706) or vice versa.

Thus, a new external, non-invasive, unisex catheter has been described. The reader should note however that the claims set forth below and the full scope of their equivalents are what define the scope of the invention.

1. A non-invasive unisex external catheter comprising:
a dressing element configured to attach to a user;
a body which is attached to said dressing element;
a flange attached around a perimeter of said body, wherein said flange is configured to attach to said dressing element; and
a suction element, wherein said suction element is inserted inside said body, said suction element having at least one hole for urine drainage.

2. The suction element of claim 1, wherein said suction element is configured to guide a stream of urine outside said body, wherein said stream of urine is collected for disposal.

3. The flange of claim 1, wherein said flange has a peelable layer, wherein an adhesive layer remains once said peelable layer is removed.

4. The dressing element of claim 1, wherein said dressing pad has a peelable layer, wherein said adhesive layer remains once said peelable layer is removed.

5. The flange of claim 1, said flange having a slit configured to allow said suction element to be inserted inside said body.

6. The body of claim 1, wherein said body has a concave shape.

7. The dressing element of claim 1, wherein said dressing element is configured to attach to said user via an adhesive.

8. The dressing element of claim 1, wherein said dressing element is a breathable material.

9. The flange of claim 1, wherein said flange is a breathable material.

10. The body of claim 1, wherein said body is pliable, wherein said pliability enables said body to contour to a plurality of said users.

11. A non-invasive unisex external catheter comprising:
a dressing pad, wherein said dressing pad is configured to attach to a user via an adhesive;
a body having a hollow concave shape, said body being configured to sit on said dressing pad;
a flange, wherein said flange is attached around a perimeter of said body, wherein said flange is attached to said dressing pad;
a slit, wherein said slit is located on said flange; and
a tube, wherein said tube is inserted inside said body via said slit, said tube having at least one hole for urine drainage, wherein said urine is drained through said at least one hole for disposal outside said body.

12. The flange of claim 11, wherein said flange has a peelable layer, wherein an adhesive remains once said peelable layer is removed.

13. The dressing pad of claim 11, wherein said dressing pad has a peelable layer, wherein said adhesive remains once said peelable layer is removed.

14. The flange of claim 11, said flange has a slit configured to allow said tube to be inserted inside said body.

15. The dressing pad of claim 11, wherein said dressing pad is a breathable material.

16. The flange of claim 11, wherein said flange is a breathable material.

17. The body of claim 11, wherein said body is pliable, wherein said pliability enables said body to contour to a plurality of users.

18. A method of attaching a non-invasive unisex external catheter on a user comprising:
peeling off a peelable layer of a dressing element;
attaching said dressing element on a user by positioning said dressing element around a genital area of said user;
pressing said dressing element against said user to establish a seal around said genital area of said user;
peeling off a peelable layer of a flange;
attaching bottom end of said flange to said dressing element;
placing body over said genital area of said user;
pressing on bottom end of said flange, wherein said pressing said bottom end of flange enables a seal between said dressing element and said bottom end of flange;
wrapping suction element around said user's genitals, wherein said suction element forms a loop around said user's genitals;
inserting a suction element through a slit located on said flange thereby placing said suction element inside said body;
attaching top end of said flange to said dressing element; and
pressing on top end of said flange, wherein said pressing said to end of flange enables a seal between said dressing element and said top end of flange.

19. The method of claim 18, wherein said flange has a peelable layer wherein an adhesive layer remains once said peelable layer is removed.

20. The method of claim 18, wherein said dressing element has a peelable layer, wherein said adhesive layer remains once said peelable layer is removed.
